SEQUENCE LISTING (5 sequences)

SEQ ID NO: 1 - protein, 254 aa, Homo sapiens
  MISC_FEATURE (176)..(176): V or F
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLXGSKNVSSETVNITITQGLAVSTISSFFPPGYQVSFCLVMVLLFAVDTGLYFSVKTNIRSSTRDWKDHKFKWRKDPQDK

SEQ ID NO: 2 - protein, 236 aa, Homo sapiens
  MISC_FEATURE (158)..(158): V or F
RTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLXGSKNVSSETVNITITQGLAVSTISSFFPPGYQVSFCLVMVLLFAVDTGLYFSVKTNIRSSTRDWKDHKFKWRKDPQDK

SEQ ID NO: 3 - DNA, 22 bp, Artificial Sequence
  FcgR3A sense
tccaaaagccacactcaaagtc

SEQ ID NO: 4 - DNA, 21 bp, Artificial Sequence
  FcgR3A reverse
agacacatttttactcccatc

SEQ ID NO: 5 - DNA, 68 bp, Artificial Sequence
  FcgR3A reverse allele F
gcgggcagggcggcgggggcggggccggtgatgttcacagtctctgatcacacatttttactcccata

(19) United States
(12) Patent Application Publication
(10) Pub. No.: US 2018/0246109 A1
(43) Pub. Date: Aug. 30, 2018

(54) LYMPHOID HEMOPATHY PROGNOSIS METHOD

(71) Applicants: Université de Montpellier, Montpellier (FR); Centre Hospitalier Universitaire De Montpellier, Montpellier Cedex 5 (FR); Centre National de la Recherche Scientifique (CNRS), Paris (FR); Institut National de la Sante et de la Recherche Medicale (Inserm), Paris (FR)

(72) Inventors: Guillaume Cartron, Saint Christol (FR); Edouard Tuaillon, Castelnau-le-lez (FR); Anne-Laure Gagez, Montpellier (FR); Renaud Cezar, Clarensac (FR)

(21) Appl. No.: 15/735,433

(22) PCT Filed: Jun. 9, 2016
(86) PCT No.: PCT/FR2016/051388

§ 371 Date: Dec. 11, 2017

(30) Foreign Application Priority Data
Jun. 9, 2015 (FR) 1555253

(51) Int. Cl. G01N 33/574

(52) U.S. Cl. CPC G01N 33/57426; G01N 2333/70596; G01N 2333/5428

(57) ABSTRACT

A prognosis method including determining the quantity of B lymphocyte cells secreting interleukin 10 in a biological sample, such that a patient from whom the tumor sample is taken having a quantity of B cells secreting IL-10 below 5% will have more than a 50% chance of a good prognosis after a treatment.

### REFERENCE TO A SEQUENCE LISTING

The instant application contains a Sequence Listing which has been submitted in ASCII format via EFS-Web and is hereby incorporated by reference in its entirety. Said ASCII copy, created on Jan. 18, 2018, is named 04475 P0008A sequence listing.txt and is 5,595 bytes in size.

### FIELD OF INVENTION

The present invention relates to a prognosis method for lymphoid blood diseases, in particular lymphoid leukemias and lymphomas.

### BACKGROUND OF THE INVENTION

Monoclonal antibodies have revolutionized therapeutic care in a large number of pathologies, including cancers and immune diseases. Rituximab (MabThera®) has for example made it possible to improve survival for patients with a lymphoma or a chronic lymphoid leukemia and the quality of life for patients with immune disease.

Rituximab is a chimeric immunoglobulin IgG1 recognizing CD20 expressed by the B lymphocytes from the pro-lymphocyte stage to the plasma cell stage. Different action mechanisms of rituximab have been described in vitro, some depending on its variable portion Fv (apoptosis), the others on its constant portion Fc (ADCC, CDC, phagocytosis). FcγRIIIa is expressed by the natural killer cells (NK cells) and the macrophages, effector cells of antibody-dependent cellular cytotoxicity (ADCC).

It has been shown that a polymorphism of the FCGR3A gene leading to a substitution of amino acid 158 on the (FcγRIIIa-158VF) receptor was making it possible to differentiate between patients responding well (158-V homozygote) to rituximab and patients not responding as well (158-F homozygote and heterozygote).

In particular, it is known from patent application WO2003035904 that the determination of the FCGR3A genotype of a subject can be used for patients suffering from malignant tumors, in particular lymphomas, and is appropriate for the selection of the best-responding subjects and/or adjusting treatment conditions or protocols for subjects responding with a low response profile.

Nevertheless, the action mechanisms of in vivo anti-CD20 remain poorly understood and may differ based on the pathology. Furthermore, it is important to note that the prediction of the efficacy by FcγRIIIa-158VF polymorphism analysis is not currently clinically used, since 67% of patients with an unfavorable phenotype still respond to rituximab. There is therefore an interest in better understanding the action mechanisms in order to propose new strategies for improving treatment, but also to better select the patients who may not respond to this type of expensive treatment so as to steer them toward other therapeutic alternatives.

The immune cells include B lymphocytes secreting interleukin-10 (IL-10) (B10 cells), an immunomodulatory cytokine in particular serving to limit the cytotoxic T response. These cells are present in mice and humans. Tumor B lymphocytes may have a B10 function, i.e., secrete IL-10.

It has been shown in a murine lymphoma model that the frequency of B10 in mice was interfering with the response to treatment using a murine anti-CD20 (Horikawa et al. J Clin Invest. 2011 Nov. 1; 121(11): 4268-4280). However, the frequency of the B10 cells in humans has not been linked to the therapeutic response of patients treated with an anti-CD20 antibody, since the study was conducted in mice. Furthermore, the biological activity of the various immunoglobulin isotypes as well as the expression of the receptors at the Fc portion of the immunoglobulins are very different between humans and mice. The extrapolation of the results obtained in mice to humans is therefore not possible, and in no case are the results obtained in a murine model applicable to humans without clinical demonstration.

Understanding the response to immunotherapy remains, and it is necessary to have elements making it possible to adapt the treatment to patients.

### SUMMARY OF THE INVENTION

One of the aims of the invention is to offset these drawbacks.

Another aim of the invention is to provide a prognosis method enabling a quick and easy determination of the therapeutic protocol to be applied in a given pathological situation, taking into account the likelihood of success and in order to limit costs related to therapeutic failures.

Still another aim of the invention is to provide a prognosis kit making it possible to carry out said method.

The invention relates to an in vitro prognosis method for the therapeutic response, mediated by a therapeutic antibody, in a tumor sample taken from a patient with a blood disease,

said antibody being an antibody depleting the cells of said blood disease,

the method comprising the in vitro determination of the quantity of B lymphocyte cells secreting interleukin 10 in said tumor sample,

such that a patient from whom the tumor sample is taken, having a quantity of B cells secreting IL-10 below 5% of the total cell quantity of said sample, will have more than a 50% chance of experiencing a depletion of more than 90% of the cells of said blood disease after treatment with said therapeutic antibody.

The invention is based on the surprising observation made by the inventors on the one hand that the aforementioned blood diseases have a population of B cells secreting IL-10, and on the other hand that the quantity of these cells affects the prognosis of the patient if he is treated with a therapeutic antibody. Furthermore, the inventors have noted that the lower the quantity of cells secreting IL-10 is before treatment, the better the response to treatment will be, and therefore the better the patient's prognosis will be.

In particular, the inventors have been able to show that if the quantity of cells secreting IL-10 is below 5% of the total number of circulating cells of the blood disease, the patient's prognosis after treatment will be favorable in more than half of cases.

In the context of the invention, “tumor sample from a patient with a blood disease” refers to a blood sample, or a biopsy, for which all or part of the sample or biopsy comprises tumor cells. The aforementioned tumor sample is obtained from the patient before beginning the treatment seeking to eradicate the blood disease.

Erythrocytes, anucleates, are not taken into consideration.

The prognosis method according to the invention relates to therapeutic antibodies, or more generally “antibodies.” Both names will be used below, unless otherwise specified. A “therapeutic antibody” refers to an antibody whose function is to eliminate determined target cells in an individual or a patient. Examples of target cells are tumor cells, cells infected by one or more viruses, pathological immunocompetent cells (for example B or T lymphocytes, antigen-presenting cells, etc.) involved in allergies, autoimmune disease, etc., or even normal cells (in case of endothelial cells in an anti-angiogenic therapeutic strategy). The preferred target cells are tumor cells and infected cells.

Therapeutic antibodies with isotype IgG1 like rituximab can mediate an antibody-dependent cellular cytotoxicity, or ADCC, as well as an antibody-dependent cellular phagocytosis, or ADPC, and a complement dependent cytotoxicity, or CDC, lysis.

The therapeutic antibodies according to the invention can be produced by hybridomas or genetic engineering, and advantageously have isotype IgG1 or IgG3. The therapeutic antibodies preferred in the invention are antibodies with the aforementioned isotype against tumor antigens (molecules expressed on the surface of tumors) such as CD20, CD22, CD25, CD38, or more generally hematopoietic tumor antigens, preferably lymphoids.

A “depleting” antibody refers, in the invention, to an antibody that is capable of causing the depletion of the target cells, i.e., their reduction or depletion.

The prognosis of the invention is based on the determination of the quantity of B cells of the tumor that are capable of secreting interleukin 10, or IL-10. IL-10 has an inhibiting effect on the immune response, by inhibiting the production of certain cytokines, such as interleukin 2, interleukin 3, TNF and certain interferons. IL-10 also acts on the immunity by modulating the number of the different cells involved in the immune system (mastocytes, lymphocytes, etc.).

By quantifying the number of B lymphocytes secreting IL-10 in blood diseases, the inventors have observed that tumors having fewer than 5% of these cells before treatment responded better to treatment with a therapeutic antibody, which leads to a better prognosis for the patient. It is then possible to consider therapy for the patient based on this therapeutic antibody alone, or in combination with other protocols (chemotherapy, radiation, etc.). Beyond the threshold of 5% B lymphocytes secreting IL-10, the prognosis of the patient treated with a single therapeutic antibody will probably not be very good, and it is therefore recommended to consider another, more appropriate therapeutic regimen.

Consequently, the prognosis of the invention makes it possible to determine the likelihood of success of a treatment using a therapeutic antibody, and to determine the best therapy to offer the patient, while streamlining costs, avoiding giving cumbersome and expensive treatments to patients who will not respond well, or not at all.

Advantageously, the invention relates to the aforementioned prognosis method, which further comprises the determination of the amino acid in position 176 of the sequence of the FcγRIIIa receptor, as shown by sequence SEQ ID NO: 1, or in position 158 of the sequence of the FcγRIIIa receptor, as shown by sequence SEQ ID NO: 2,

such that a patient, from whom the tumor sample is taken,
having a valine in position 176 of the sequence of the FcγRIIIa receptor, as shown by sequence SEQ ID NO: 1, or in position 158 of the sequence of the FcγRIIIa receptor, as shown by sequence SEQ ID NO: 2, and
having a quantity of B cells secreting IL-10 in the sample below 5% of the total cells of the sample before treatment

will have more than a 50% chance of experiencing a depletion of more than 90% of the cells of said blood disease after treatment with said therapeutic antibody.

In other words, the invention relates to an in vitro prognosis method for the therapeutic response, mediated by a therapeutic antibody, in a tumor sample taken from a patient with a blood disease, said antibody being an antibody depleting the cells of said blood disease, the method comprising the in vitro determination in the cells of said sample:
of the amino acid in position 176 of the sequence of the FcγRIIIa receptor, as shown by sequence SEQ ID NO: 1, or in position 158 of the sequence of the FcγRIIIa receptor, as shown by sequence SEQ ID NO: 2, and
of the quantity of B lymphocyte cells secreting interleukin 10 before treatment,

such that a patient, from whom the tumor sample is taken,
having a valine in position 176 of the sequence of the FcγRIIIa receptor, as shown by sequence SEQ ID NO: 1, or in position 158 of the sequence of the FcγRIIIa receptor, as shown by sequence SEQ ID NO: 2, and
having a quantity of B cells secreting IL-10 in the sample below 5% of the total cells of the sample
will have more than a 50% chance of experiencing a depletion of more than 90% of the cells of said blood disease after treatment with said therapeutic antibody.

The inventors have made a second surprising observation: if one combines the determination of the quantity of B lymphocytes secreting IL-10 with the determination of a particular polymorphism of the FcγRIIIa receptor, the patient's prognosis will be significantly improved as a result.

Indeed, as shown by the example below, the determination of only the polymorphism of the FcγRIIIa receptor does not make it possible to have sufficient information to predict a correct response by patients to treatment with a therapeutic antibody.

The polymorphism of the FcγRIIIa receptor has already been described in the state of the art, and in particular in international application WO2003035904. Depending on whether the amino acid in position 176 of the protein, or in position 158 of the mature protein having undergone a modification, is a phenylalanine (F) or a valine (V), the therapeutic response to a therapeutic antibody will be different.

In the invention, the protein made up of sequence SEQ ID NO: 1 corresponds to the FcγRIIIa protein, and the protein made up of sequence SEQ ID NO: 2 corresponds to the mature FcγRIIIa protein.

Man being diploid, there are therefore three possible genotypes regarding this polymorphism: 176V/V (or 158V/V), 176V/F (or 158V/F) and 176F/F (or 158F/F).

In the context of the aforementioned method, the individuals or patients with a valine in position 176, or 158, on both of their alleles of the FCGR3A gene are considered to be V/V homozygous patients. Thus, a patient who is V/V homozygous in position 176 (158) of the FcγRIIIa receptor and who has a level of B lymphocytes secreting IL-10 below 5% relative to the total cells of the tumor before treatment will have more than a 50% chance of responding well to treatment with therapeutic antibody, and having a depletion of more than 90% of the tumor cells after treatment.

It is advantageous for the level of B cells secreting IL-10 to be less than 5% of the total quantity of cells in the tumor. This means that overlaps of 4.9%, 4.8%, 4.7%, 4.6%, 4.5%, 4.4%, 4.3%, 4.2%, 4.1%, 4%, 3.9%, 3.8%, 3.7%, 3.6%, 3.5%, 3.4%, 3.3%, 3.2%, 3.1%, 3%, 2.9%, 2.8%, 2.7%, 2.6%, 2.5%, 2.4%, 2.3%, 2.2%, 2.1%, 2%, 1.9%, 1.8%, 1.7%, 1.6%, 1.5%, 1.4%, 1.3%, 1.2%, 1.1%, 1%, 0.9%, 0.8%, 0.7%, 0.6%, 0.5%, 0.4%, 0.3%, 0.2% or 0.1% of cells secreting IL-10 are particularly advantageous.

Advantageously, the invention relates to the aforementioned prognosis method, in which the blood disease is a blood disease whose cells express the CD20 surface marker, i.e., whose tumor cells express the CD20 surface marker.

CD20 is the first differentiation antigen for human B lymphocytes to have been identified using monoclonal antibodies. It is a specific marker of B cells over the course of their development from the pre-B stage to the mature B lymphocyte stage. It is nevertheless missing from the surface of the plasma cells. The coding gene is located in chromosome 11. CD20 belongs to a family of molecules comprising CD20, the β chain of the high affinity receptor for IgE and the HTm4 molecule present on the surface of the lymphoid and myeloid hematopoietic cells and whose function is unknown.

In one advantageous embodiment, the invention relates to the aforementioned prognosis method, in which the blood disease is chosen from among B-cell chronic lymphoid leukemia (CLL), diffuse large B cell lymphomas and all of these histological variants expressing CD20, follicular lymphomas and mantle cell lymphomas, marginal zone lymphomas, MALT (mucosa-associated lymphoid tissue) lymphomas, Burkitt lymphomas, lymphoplasmacytic lymphomas, Waldenström's disease, B-cell prolymphocytic leukemia, and all unclassifiable CD20+ B-cell lymphomas.

Consequently, advantageously, the invention relates to an in vitro prognosis method for the therapeutic response, mediated by a therapeutic antibody, in a tumor sample taken from a patient with a chronic lymphoid leukemia.

In one advantageous embodiment, the invention relates to a prognosis method as defined above, in which the quantity of B-cells secreting IL-10 in the sample is measured by flux cytometry, after activating the secretion of IL-10, or by measuring a circulating IL-10 level in the serum.

Although several means exist for determining the quantity of B-cells secreting IL-10 in the tumor sample, the advantageous method of the invention consists of detection by flux cytometry.

To that end, the nucleated cells isolated from a patient are placed in culture and treated with compounds stimulating IL-10 secretion. It is then possible either to determine the quantity of IL-10 secreted in the culture medium, which is not good quantitative information given that the stimulation is artificial, or to carry out a secretion-blocking step, such that the produced IL-10 is sequestered in the secreting B-cells. The cells can then be marked with an IL-10 antibody, and quantified by flux cytometry. Experimental details are given in the example below.

The inventors have also shown that a correlation existed between the quantity of B-cells secreting IL-10 in the tumor of a patient with a blood disease as defined above and the quantity of IL-10 circulating in the plasma of said patients (see also example below). Consequently, by a simple assay of the IL-10 in the serum, it is possible to determine, at least more approximately, the proportion of B-cells secreting IL-10 in the patient.

In another advantageous embodiment, the invention relates to the aforementioned prognosis method, in which the in vitro determination of the amino acid in position 176 of the sequence of the FcγRIIIa receptor, as shown by sequence SEQ ID NO: 1, or in position 158 of the sequence of the FcγRIIIa receptor, as shown by sequence SEQ ID NO: 2 is done by polymerase chain reaction (PCR), such as PCR, PCR coupled with reverse transcription (RT-PCR) or nested PCR, using conventional methods and specific oligonucleotides (primers).

The genotyping of the gene coding the FcγRIIIA receptor can be done by different techniques comprising the analysis of the nucleic acids or proteins. The analyses can be done by specific digestion by restriction enzymes, hybridations, or advantageously amplification/separation/sequencing. It is even possible to consider determining and identifying the polymorphism from RNAs. The techniques described in application WO2003035904 apply mutatis mutandis.

In the context of the use of amplification reaction, such as PCR techniques, the oligonucleotides used as primers will preferably be single-strand oligonucleotides comprising 50 nucleotides, advantageously about 30 nucleotides, preferably 17 to 25 nucleotides. It is preferable for the oligonucleotides to have at least 5, or even at least 8 nucleotides strictly complementary to the target sequence to be amplified, and in particular the nucleotide in position 3′ of said oligonucleotide. To determine advantageous oligonucleotides, one skilled in the art may use the sequence of the human FCGR3A gene, accessible in the databases, in particular under number AL590385 in the GenBank database, or the sequence of the complementary DNA accessible in the GenBank database under number NM 000569.

Particularly advantageous nucleotides to determine the prognosis according to the invention are the oligonucleotides represented by sequences SEQ ID NO: 3, SEQ ID NO: 4 and SEQ ID NO: 5.

Of course, the invention is not limited to these specific oligonucleotides, and one skilled in the art is capable of conceiving of other specific oligonucleotides.

In still another advantageous embodiment, the invention relates to the aforementioned method, where the therapeutic antibody is an immunoglobulin IgG1 or IgG3, in particular a CD20 antibody, in particular rituximab.

The therapeutic antibody that is used in the context of the inventive method is therefore advantageously a CD20 antibody, and in particular rituximab. In this configuration, the inventive method advantageously aims to propose a response prognosis for patients treated with these antibodies for B-cell chronic lymphoid leukemia or B-cell lymphoma.

Rituximab is a chimeric monoclonal antibody against the CD20 surface molecule. The variable part Fab of rituximab bonds to the CD20 antigen of the B lymphocytes, and its constant part Fc can generate immune effector functions that cause the lysis of these lymphocytes. The possible mechanisms of the cell lysis induced by the effectors are complement dependent cytotoxicity (CDC), involving the bond of the C1q fragment, an antibody dependent cellular cytotoxicity (ADCC), going through one or several of the Fcγ receptors of the surface of the granulocytes, macrophages and NK cells, and a phagocytosis (or ADPC) also going through the interaction with the Fcγ receptors of the surface of the granulocytes, macrophages. It has also been demonstrated that rituximab, by bonding with the CD20 antigen of the B lymphocytes, causes cell death by apoptosis.

Consequently, in this advantageous aspect, the invention relates to an in vitro prognosis method for the therapeutic response, mediated by a CD20 therapeutic antibody with isotype IgG1 or IgG3 in a tumor sample taken from a patient with a chronic lymphoid leukemia or a B-cell lymphoma, the method comprising the in vitro determination in the cells of said sample:
of the amino acid in position 176 of the sequence of the FcγRIIIa receptor, as shown by sequence SEQ ID NO: 1, or in position 158 of the sequence of the FcγRIIIa receptor, as shown by sequence SEQ ID NO: 2, and
of the quantity of B lymphocyte cells secreting interleukin 10, such that a patient, from whom the tumor sample is taken,
having a valine in position 176 of the sequence of the FcγRIIIa receptor, as shown by sequence SEQ ID NO: 1, or in position 158 of the sequence of the FcγRIIIa receptor, as shown by sequence SEQ ID NO: 2, and
having a quantity of B cells secreting IL-10 in the sample below 5% of the total cells of the sample
will have more than a 50% chance of experiencing a depletion of more than 90% of the cells of said leukemia or said lymphoma after treatment with said CD20 therapeutic antibody.

In another aspect, the invention relates to a kit for the in vitro prognosis of a patient with a blood disease who may be treated with a therapeutic antibody, in particular for the prognosis of chronic lymphoid leukemia or a B-cell lymphoma, comprising:

means for detecting the amino acid in position 176 of the sequence of the FcγRIIIa receptor, as shown by sequence SEQ ID NO: 1, or in position 158 of the sequence of the FcγRIIIa receptor, as shown by sequence SEQ ID NO: 2,

means for determining the quantity of B lymphocyte cells secreting IL-10, and

one or several control samples.

The prognosis kit according to the invention provides the means for measuring the two parameters that are essential to the aforementioned method: the polymorphism of the receptor and the quantity of B-cells secreting IL-10. In order to normalize the prognosis, the kit also contains control samples that correspond to samples taken from healthy individuals (negative controls) and/or sick individuals with a poor prognosis (positive controls) and/or individuals having been diagnosed with a poor prognosis (positive controls).

This or these control sample(s) can also be indications or computer data on a physical medium allowing calibration of a flux cytometer to determine the quantity of B-cells secreting IL-10.

As mentioned above, the means for detecting the quantity of B-cells secreting IL-10 can be antibodies for evaluation by flux cytometry of the cells, but also drugs to stimulate and/or inhibit IL-10 production. It may also involve antibodies making it possible to measure the IL-10 level circulating in the plasma.

Advantageously, the invention relates to a prognosis kit as defined above, in which the means for detecting the amino acid in position 176 of the sequence of the FcγRIIIa receptor, as shown by sequence SEQ ID NO: 1, or in position 158 of the sequence of the FcγRIIIa receptor, as shown by sequence SEQ ID NO: 2, comprise oligonucleotides with sequences SEQ ID NO: 3, SEQ ID NO: 4 and SEQ ID NO: 5.

Of course, one skilled in the art can propose other oligonucleotides to amplify, by PCR, the region corresponding to the polymorphism, and determine which alleles are carried by the tested patient.

In another aspect, the invention relates to a computer program product on an appropriate medium for the in vitro detection of B-cells secreting IL-10 in a sample of a patient with a blood disease.

Another aspect of the invention relates to software or a computer program product designed to carry out the detection of the B-cells secreting IL-10 by flux cytometry and/or comprising portions/means/program code instructions for the execution of said detection when said program is executed on a computer, in particular connected to a flux cytometer. Advantageously, said program is comprised in a data recording medium readable by computer. Such a medium is not limited to a portable recording medium such as a CD-ROM, but may also be part of a device comprising an internal memory and a computer (for example RAM and/or ROM), or an external memory device such as hard drives or USB keys, or a nearby or remote server.

Consequently, the invention relates to a computer program product on an appropriate medium for the implementation of the prognosis method as previously defined, said computer program product enabling the determination of the quantity of B-cells secreting IL-10.

This computer program in particular makes it possible to perform automatic and normalized adjustments, such that the user then only needs to load the information in the cytometer and pass the tumor cells to obtain, reproducibly, the determination of the quantity of B-cells secreting IL-10.

In another aspect, the invention relates to a method seeking to improve the treatment of blood diseases using therapeutic antibodies comprising the use of at least one inhibitor of the secretion or activity of IL-10 by the lymphocytes.

In particular, the invention relates to a method seeking to improve the treatment of blood diseases using a CD20 antibody, in particular rituximab, comprising the use of at least one inhibitor of the secretion or activity of IL-10 by the lymphocytes.

In another aspect, the invention relates to a method seeking to improve the treatment of blood diseases using therapeutic antibodies comprising the use of at least one inhibitor of lymphocytes secreting IL-10.

In particular, the invention relates to a method seeking to improve the treatment of blood diseases using a CD20 antibody, in particular rituximab, comprising the use of at least one inhibitor of B-cells secreting IL-10.

An inhibitor of B-cells secreting IL-10, or lymphocytes secreting IL-10, refers, within the meaning of the invention, to compounds or molecules, or mixtures thereof, capable of interfering with the activity of B10 lymphocytes, either by inhibiting their ability to secrete IL-10, or by inhibiting their proliferation, or by causing their death, in particular by apoptosis.

Since the inventors have identified the influence of B10 cells on the efficacy of rituximab, and the correlation between the secretion of IL-10, it is advantageous to propose a method that indeed seeks to:
either inhibit the production of IL-10 by the B lymphocytes, for example by inhibiting synthesis or secretion,
or inhibit the biological activity of IL-10.

Secretion inhibitors such as brefeldine A can be used.

It is also possible to consider using IL-10 inhibitors such as IL-10 antibodies, or to use mimetic peptides that will saturate the targets of the interleukin and thus inhibit the action of the native protein.

One skilled in the art, with his general knowledge, can easily propose a method for screening IL-10 inhibitor molecules, or molecules inhibiting its secretion.

The invention also relates to the use of inhibitors of the activity or secretion of IL-10 for the preparation of a medicinal drug seeking to promote blood disease treatment with therapeutic antibodies, in particular an anti-CD20 or rituximab.

The invention relates to an inhibitor of the activity or secretion of IL-10 for its use in the context of the treatment of blood diseases treated with at least one therapeutic antibody, in particular a CD20 antibody or rituximab.

Advantageously, the invention relates to a composition comprising at least one inhibitor of the activity or secretion of IL-10 and at least one therapeutic antibody, in particular a CD20 antibody or rituximab, for the treatment of blood diseases whereof the tumor cells have a valine in position 176 of the sequence of the FcγRIIIa receptor, as shown by sequence SEQ ID NO: 1, or in position 158 of the sequence of the FcγRIIIa receptor, as shown by sequence SEQ ID NO: 2.

### BRIEF DESCRIPTION OF THE DRAWINGS

The invention will be better understood in light of the five figures and the example that follow:

FIG. 1 shows a graph showing that the frequency of competent IL-10 leukemia B-cells (CLL) correlates with the plasma IL-10 level on day D0, i.e., before treatment (p=0.0166, r=3969). The x-axis shows the quantity of IL-10 in the plasma in pg/mL and the y-axis shows the percentage of B10 cells expressed in %.

FIG. 2 shows a graph showing that the lymphocyte depletion observed after treatment with rituximab alone (D22) is statistically influenced by the frequency of competent IL-10 CLL B-cells on D0 (p=0.004). The x-axis shows the percentage of lymphodepletion in % and the y-axis shows the percentage of B10 cells expressed in %.

FIG. 3 shows a graph showing that the lymphocyte depletion is statistically greater for patients carrying the FcγRIIIa-158V allele.

FIG. 4 shows ROC curves (Receiver operating curve) generated by logistical regression for the percentage of competent IL-10 CLL B-cells alone (A) (AUC=0.763), the carriers of V vs F/F alleles for the FCGR3A polymorphism (B) (AUC=0.675) and a combination of the percentage of competent IL-10 CLL B-cells and carriers of V vs F/F alleles for the FCGR3A polymorphism (C) (AUC=0.855).

FIG. 5 shows a graph comparing the number of regulator B-cells (listed by cytometry—x-axis) to the assay of the IL-10 secretion (quick method—y-axis). The rectangle with broken lines corresponds to individuals with a low risk of suboptimal response with a CD20 antibody. The rectangle with solid lines corresponds to individuals with a high risk of suboptimal response with a CD20 antibody. A good correlation is observed between the results obtained by the two methods (R=0.79).

### DETAILED DESCRIPTION OF THE INVENTION

### Examples

### Example 1

The action mechanisms of rituximab (marketed under the brands MabThera®, Rituxan®) remain unknown and could be different depending on the subtype of B-cell lymphoproliferative disorder. Rituximab is known for causing in vitro apoptosis, complement-dependent cytotoxicity (CDC), antibody-dependent cellular cytotoxicity (ADCC) and antibody-dependent phagocytosis (ADPC), and some results tend to involve these mechanisms in vivo.

Certain factors affecting the response to rituximab have recently been discovered.

The inventors had previously observed that the polymorphism of the FcγRIIIa V/F receptor affects the clinical response to rituximab treatment. Since this polymorphism alters the affinity of the constant part of IgG1 for the FcγRIIIa receptor (expressed on the surface of NK cells and macrophages), the inventors put forth the hypothesis that the ADCC mechanism should be significant in the context of treating follicular lymphomas treated with rituximab.

Regulator B-cells were recently identified in humans and mice as a cellular subtype capable of secreting IL-10. These B-cells, also called B10 cells or B10, are characterized by their ability to modulate inflammation, autoimmunity and the innate or adaptive immune response based on IL-10 production. In a murine model, the B10 cells would be capable of inhibiting the elimination of lymphoma cells induced by a CD20 antibody, by acting on the monocyte functions mediated by the constant parts of the immunoglobulins (Fc parts).

More recently, it has been shown that the cells of a clonal chronic lymphoid leukemia (CLL) had immunosuppressive properties and an IL-10 competence.

The inventors then considered that the competent IL-10 cells of CLL's could affect the efficacy of treatment with rituximab in patients with this type of leukemia.

Patients and Treatment

The Patients.

A prospective and randomized phase II study was conducted in 59 French centers including 140 patients between June 2012 and January 2013. The patients not having received any prior treatment (between 18 and 65 years of age) and whose chronic lymphoid leukemia diagnosis was confirmed by immunophenotyping according to the 2008 IWCLL criteria (Binet stage C, or Binet stage A or B with an active disease), were enrolled in this trial. An additional inclusion criterion was taken into account: the absence of 17p deletion, evaluated by FISH (<10% positive cores). All patients provided informed consent in writing before inclusion.

Randomization.

The patients were stratified based on their IGVH mutational status after FISH analysis (11q deletion) and assigned randomly with a 1:1 ratio to receive either the standard dose of FCR (fludarabine, cyclophosphamide and rituximab) chemotherapy for arm A or Dense-FCR with a rituximab pre-phase before standard FCR treatment for arm B.

Treatment.

The standard FCR treatment consists of 6 rounds separated by 28 days of rituximab (375 mg/m² for the 1st round D1 and 500 mg/m² for the other rounds), fludarabine (40 mg/m²/d D2-4), cyclophosphamide (250 mg/m²/d D2-4). For the experimental arm, the FCR treatment is preceded by a rituximab pre-phase made up of 4 successive infusions, distributed as follows: 500 mg on D0, and 2000 mg on D1, D8 and D15. Round 1 begins on D22 for the patients, and the following rounds are separated by 28 days.

Identification of Competent IL-10 Cells in CLL's, IL-10 Trial and FCGR3A Genotyping

The competent IL-10 cells of the CLL's are identified by flux cytometry from purified mononucleated blood cells after polyclonal stimulation.

The peripheral blood mononucleated cells (PBMC) have been purified from patients with CLL in arm B of the study using a Ficoll-Hypaque density gradient (Eurobio, Courtaboeuf, France).

The PBMC's were resuspended (9×10⁶ cells/mL) in medium (RPMI 1640—Biotech GmbH, Aidenbach, Germany) containing 10% fetal calf serum (Eurobio, Courtaboeuf, France), 2 mM of L-glutamine (Eurobio, Courtaboeuf, France), 100 U/mL of penicillin, 100 μg/mL of streptomycin, and 2.5 μg/mL amphotericin (all antibiotics are obtained from Tebu-bio, Le Perray-en-Yvelines, France).

The clonal activation of the B lymphocytes was done with CpG (ODN 2006, 10 μg/mL; InvivoGen, San Diego, USA), CD40L (50 ng/mL; R&D Systems, Minneapolis, Minn., USA) and anti-polyHistidine (500 ng/mL; R&D Systems, Minneapolis, Minn., USA) for 48 hours at 37° C. in a humid atmosphere comprising a 5% CO₂−95% air mixture. Phorbol myristate acetate (PMA, 50 ng/mL; Sigma-Aldrich, St. Louis, Mo., USA) and ionomycin (1 μg/mL; Sigma-Aldrich, St. Louis, Mo., USA) were added to stimulate IL-10 production. After 4 h at 37° C. in a humid atmosphere comprising a 5% CO₂-95% air mixture, brefeldin A (1×solution/mL; BioLegend, San Diego, Calif., USA) was added to block the IL-10 secretion in order to identify the B10 cells. The cells were marked using the following antibodies: anti CD19 BV421 (HIB 19), anti CD69 PE/Cy7 (FN 50), anti CD38 APC (HIT 2), anti IL-10 PE (JES3-9D7) from BioLegend (San Diego, Calif., USA), and anti CD45 KO (J.33) and anti CD5 FITC (BL1a) from Beckman Coulter (Brea, Calif., USA). The clonal CLL cells were identified as being CD19+CD5+CD20int lymphocytes. The analyses were done using a CyAn™ ADP flux cytometer (Beckman Coulter, Brea, Calif., USA).

The plasma IL-10 level was determined using the LUMINEX® technology on magnetic balls, through a laser double reading system, according to the manufacturer's recognitions (R&D Systems, Minneapolis, USA). The plasma of the 68 patients was incubated with superparamagnetic balls covered with IL-10 antibodies for 2 hours, at ambient temperature, diluted by half. The plasma IL-10 is quantified owing to the combination of two antibodies for detection: an IL-10 biotinylated antibody and a streptavidin antibody conjugated with phycoerythrin.

The FcγRIIIA-158VF polymorphism is determined by nested PCR. Briefly, single-step allele-specific multiplex PCR tests were carried out as described in Dall'Ozzo et al. (Dall'Ozzo et al. J Immunol Methods. 2003; 277(1-2):185-192) with some modifications. The 25 μL of reaction mixture comprises genomic DNA, 400 nM of forward primer (5′-TCCAAA AGCCACACTCAAAGTC-3′ (SEQ ID NO: 3)), 400 nM of reverse primer specific to allele V (5′-AGACACATTTTTACTCCCATC-3′ (SEQ ID NO: 4)) and 200 nM of reverse primer specific to allele F (5′-GCGGGCAGGGCGGCGGGGGCGGGGCCGGTGATGTTCACAGTCTCTGA TCACACATTTTTACTCCCATA-3′ (SEQ ID NO: 5)), 400 μM of each nucleotide (dNTP), 2 mM of MgCl₂ and 0.5 U of Taq DNA polymerase in its buffer (Promega, Madison, USA). The PCR conditions are: 3.5 min at 95° C. followed by 35 cycles, each cycle consisting of 95° C. for 20 sec, 56° C. for 20 sec, 72° C. for 30 sec. After amplification, the PCR products (137 bp for allele F and 81 bp for allele B) are separated on 8% acrylamide gel (Invitrogen, Carlsbad, USA) and viewed by marking with ethidium bromide.

Statistical Analyses

The data distributions were tested using the Shapiro-Wilk test. The χ² and Fisher tests were used for categorical data.

The comparison of the medians was done using a Student T test or a Mann-Whitney test.

All of the variables with a value p<0.10 in the univariate analysis are included in an intermediate model. The variables of the final model were determined using the progressive elimination method using a Student T test (p<0.05 as significant model).

All of the statistical analyses were done by implementing, at a level a, 0.05 of a bilateral test using version 3.0.2.10 of the R software.

Results and Discussion

The lymphocyte depletion after monotherapy with rituximab was evaluated on day 22 (D22) to study the influence of IL-10 competent leukemia cells (B10) on the in vivo efficacy of rituximab, in 68 patients included in the experimental arm of the study.

The median of the number of leukemia lymphocytes before the four doses of rituximab (D0) was 91.13 G/L (range: 3.74-497.40) and was 2.60 G/L (range: 0.14-189.40) at the end of the pre-phase for treatment with rituximab alone (D22). Thus, the median lymphocyte depletion after the pre-phase for treatment with rituximab alone (D22) was 95.1% (range 77.0-99.9), among which 66% obtained a depletion of more than 90%.

The characteristics of the patients and their distribution based on 90% lymphodepletion are presented in table 1 below.

No significant correlation was found between 90% lymphodepletion and the clinical parameters of age, sex, Binet stage, IGHV mutation, cytogenetic anomaly or the β2 microglobulin.

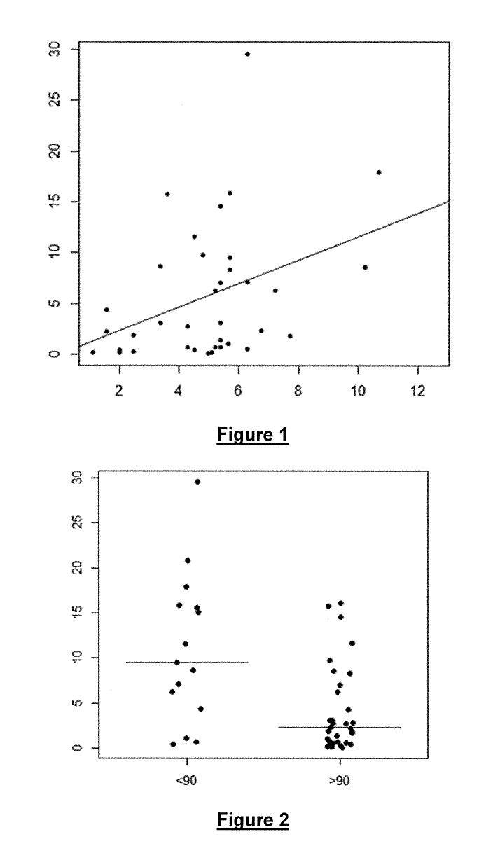

A subgroup of B10 cells was identified in all of the tested patients (n=47, median: 3.06% of CLL cells, range: 0.12 to 29.55). The frequency of B10 cells among the leukemia cells correlates with the quantity of plasma IL-10 (FIG. 1, r=0.39, p=0.02), while the plasma IL-10 level does not correlate with the non-B10 leukemia cells (not shown). The frequency of B10 cells does not vary based on the characteristics of patients, and is also not significantly different in patients with CLL with or without IGVH mutation (median: 6.29%, range: 0.12 to 15.83 versus median: 1.85%, range: 0.23 to 20.81, respectively). Furthermore, such B10 cell frequencies also do not correlate with the cytogenetic alterations (del11q, del13q, trisomy 12).

A univariate analysis shows that the frequency of B10 cells affects the lymphodepletion above 90% after the pre-phase for treatment with rituximab alone (D22) (FIG. 2, p=0.004). The frequency of B10 cells also makes it possible to predict the 3 month clinical response (complete response) after the end of treatment with the fludarabine-cyclophosphamide combination and rituximab, considered to be the reference treatment for patients with chronic lymphoid leukemia (p=0.04).

These results show that all CLL patients have a subpopulation of B10 cells that represent a variable percentage of leukemia B-cells and that have a chemically significant in vivo inhibiting effect on the activity of rituximab.

Since the FcγRIIIa-158V/F polymorphism correlates with the in vivo efficacy of rituximab in follicular lymphomas, the inventors have determined this polymorphism in the various patients of the cohort.

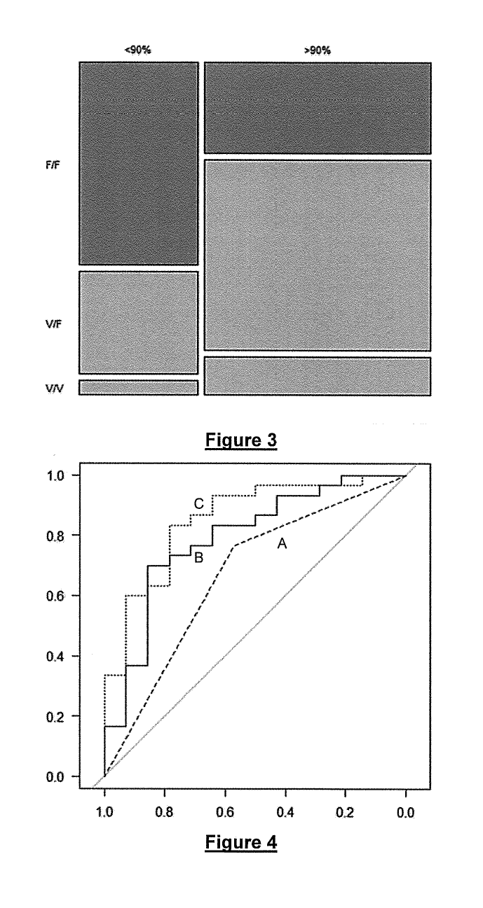

The FcγRIIIa-158V/F polymorphism is significantly associated with a normal number of lymphocytes (<5 G/L) (p=0.03) and a lymphodepletion of 90% (p=0.03) at D22. This is also the case for carriers of the FcγRIIIa-158V polymorphism (p=0.01) (FIG. 3).

Conversely, the FcγRIIIa-158V/F polymorphism does not correlate with the 3 month clinical response after immuno-chemotherapy. Consequently, these results suggest that the immune functions mediated by FcγRIIIa play a critical role in the activity of rituximab, but that the involvement of the FcγRIIIa-158V/F polymorphism could be hidden either by the high activity of the immuno-chemotherapy, or by a direct inhibition of the effector immune cells by chemotherapy.

Logistical regression analyses show that only the frequency of B10 cells and the FcγRIIIa-158V/F polymorphism are associated with a 90% lymphodepletion after the pre-phase for treatment with rituximab alone (D22) (close relative risk (OR)=0.83; confidence interval (CI): 0.72-0.3; p=0.002, and OR=4.95; 95% CI: 1; 07-27.48; p=0.04, respectively).

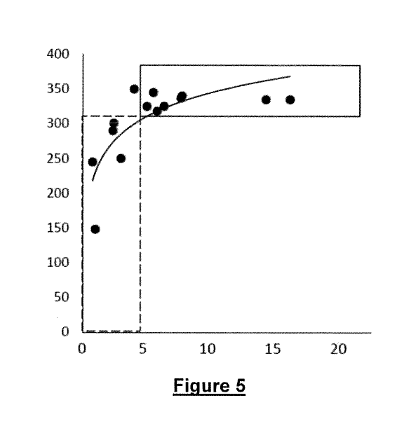

The receiver operating curve (ROC) built using the frequencies of the B10 cells and the FcγRIIIa-158V/F polymorphism shows a very significant area under the curve (AUC) (AUC=0.855; 95% CI: 0.732-0.978), leading to a good distinction between patients who have a depletion of their lymphocytes of more than 90% and those who do not have such a depletion (FIG. 4).

From the ROC curve (FIG. 4), the following data can be extracted:

Data obtained from genotyping of the FcγRIIIa receptor alone

Based on the population used during the multivariate analysis (n=44) (Area under the curve (AUC=0.669), 95% CI=0.514-0.824, the obtained results are as follows:

TABLE 2

| | Lymphodepletion > | Lymphodepletion < |
|---|---|---|
| | 90% | 90% |
| Probability > 0.5 of obtaining | 30 | 14 |
| a lymphodepletion > 90% | (true positives) | (false positives) |
| Probability < 0.5 of obtaining | 0 | 0 |
| a lymphodepletion > 90% | (false negatives) | (true negatives) |

Sensitivity = 100%
Specificity = 0%
Legend of table 2. Sensitivity and specificity for a lymphodepletion of 90% obtained by using the aforementioned computed probabilities.

One can see that a large number of false positives are identified (about 33%).

1—Data obtained from the number of B10 cells alone
a. With a threshold of 4.34%

Based on the population used during the multivariate analysis (n=44) (AUC=0.779), 95% CI=0.652-0.905, the obtained results are as follows:

TABLE 3

| | Lymphodepletion > | Lymphodepletion < |
|---|---|---|
| | 90% | 90% |
| Probability > 0.5 of obtaining | 21 | 2 |
| a lymphodepletion > 90% | (true positives) | (false positives) |
| Probability < 0.5 of obtaining | 9 | 12 |
| a lymphodepletion > 90% | (false negatives) | (true negatives) |

Sensitivity = 70.00%
Specificity = 85.71%
Legend of table 3: identical to the legend of table 2.
b. With a threshold of 3%

Based on the population used during the multivariate analysis (n=44) AUC=0.729, 95% CI=0.598-0.589, the obtained results are as follows:

TABLE 4

| | Lymphodepletion > | Lymphodepletion < |
|---|---|---|
| | 90% | 90% |
| Probability > 0.5 of obtaining | 18 | 2 |
| a lymphodepletion > 90% | | |
| Probability < 0.5 of obtaining | 12 | 12 |
| a lymphodepletion > 90% | | |

Sensitivity = 60.00%
Specificity = 85.71%
Legend of table 4: identical to the legend of table 2.
c. With a threshold of 5%

Based on the population used during the multivariate analysis (n=44) AUC=0.726, 95% CI=0.586-0.866, the obtained results are as follows:

TABLE 5

| | Lymphodepletion > | Lymphodepletion < |
|---|---|---|
| | 90% | 90% |
| Probability > 0.5 of obtaining | 21 | 3 |
| a lymphodepletion > 90% | | |
| Probability < 0.5 of obtaining | 9 | 11 |
| a lymphodepletion > 90% | | |

Sensitivity = 70.00%
Specificity = 78.57%
Legend of table 5: identical to the legend of table 2.

One can see that by imposing the threshold at less than 5% B10 cells, the false positive level falls to about 5%. B10 detection significantly improves the prognosis relative to the polymorphism.

2—Data obtained from genotyping of the FcγRIIIa receptor and B10

Based on the population used during the multivariate analysis (n=44) (Area under the curve (AUC=0.0855), 95% CI=0.732-0.978, the obtained results are as follows:

TABLE 6

| | Lymphodepletion > | Lymphodepletion < |
|---|---|---|
| | 90% | 90% |
| Probability > 0.5 of obtaining | 26 | 5 |
| a lymphodepletion > 90% | | |
| Probability < 0.5 of obtaining | 4 | 9 |
| a lymphodepletion > 90% | | |

Sensitivity = 86.67%
Specificity = 64.29%
Legend of table 6: identical to the legend of table 2.

In the preceding tables 2 to 6, the sensitivity is the measurement of the test's ability to identify a condition when it is present. It is the proportion of true positives (TP) that is detected by the test as having the “condition” [Se=TP/(TP+FN)]. The false negatives are the patients having the “condition”, but not detected by the test. A high sensitivity is preferred in screening, since it is easier to reject patients not having the condition.

The specificity is the test's ability to exclude a condition when it is absent. It is the proportion of true negatives (TN) that is detected by the test as not having the “condition” [Sp=TP/(TP+FP)]. The false positives are the patients not having the “condition,” but detected by the test as being positive.

By combining the detection of the polymorphism and the number of B10 cells, one sees an improvement in the prognosis, and in particular a very significant reduction in the number of patients considered to be false negatives. Furthermore, the double detection makes it possible to obtain, for thresholds below 5% of B10, a good sensitivity (around 86%).

The inventors have therefore determined a B10 population in patients with chronic lymphoid leukemia and receiving rituximab. At the same time, they have determined the FcγRIIIa-158VF phenotype of these patients. They have thus been able to show that the frequency of B10 cells and the FcγRIIIa-158VF polymorphism were influencing the response to rituximab in patients with chronic lymphoid leukemia. The analysis of these two factors combined makes it possible to improve the sensitivity and specificity of prediction of the prognosis response.

TABLE 1

| | Lymphodepletion | | Lymphodepletion | | Univariate | | Multivariate | | |
|---|---|---|---|---|---|---|---|---|---|
| | >90% (n = 44) | | ≤90% (n = 23) | | analysis | | analysis | | |
| | | Median | | Median | OR | Value | AUC | OR | Value |
| | N (%) | (IQR) | N (%) | (IQR) | [95% CI] | of p | [95% CI] | [95% CI] | of p |
| Age (years) | 44 (100.00) | 55.72 | 23 (100.00) | 53.99 | — | 0.792 | — | | |
| | | (51.31-58.12) | | (52.07-57.41) | | | | | |
| men | 31 (70.45) | — | 18 (78.26) | — | 0.68 | 0.693 | — | — | — |
| | | | | | [0.19-2.16] | | | | |
| Binet AB stage | 36 (81.82) | — | 16 (69.57) | — | 0.51 | 0.404 | — | — | — |
| | | | | | [0.15-1.73] | | | | |
| ECOG 0 | 31 (70.45) | — | 5 (21.74) | — | 1.50 | 0.572 | — | — | — |
| | | | | | [0.41-6.29] | | | | |
| IGHV on | 25 (56.82) | — | 16 (72.73) | — | 0.50 | 0.324 | — | — | — |
| mutated | | | | | [0.15-1.50] | | | | |
| Cytogenetic anomalies | 18 (51.43) | — | 8 (44.44) | — | 1.31 | 0.848 | — | — | — |
| Del(13q) | | | | | [0.41-4.29] | | | | |
| Del(11q) | 7 (16.67) | — | 6 (26.09) | — | 0.57 | 0.560 | — | — | — |
| | | | | | [0.16-2.07] | | | | |
| Trisomy 12 | 2 (6.06) | — | 2 (14.29) | — | 0.40 | 0.572 | — | — | — |
| | | | | | [0.03-6.04] | | | | |
| β2 | 39 (88.64) | 2.90 | 22 (95.65) | 2.76 | — | 0.857 | — | — | — |
| microglobulin | | (2.33-3.66) | | (2.33-4.29) | | | | | |
| (mg/L) | | | | | | | | | |
| CD38+ (%) | 34 (77.27) | 2.00 | 17 (73.91) | 10.50 | — | 0.133 | | — | — |
| | | (0.00-23.00) | | (1.75-26.00) | | | | | |
| Competent | 32 (72.73) | 2.30 | 15 (65.22) | 9.51 | — | 0.004 | 0.763 | 0.83 | 0.002 |
| IL-10 cells (%) | | (0.68-6.47) | | (5.35-15.70) | | | [0.604-0.921] | [0.72-0.93] | |
| FCGR3A | | | | | — | 0.028 | — | — | — |
| V/V | 5 (11.91) | — | 1 (4.55) | — | | | | | |
| V/F | 25 (59.52) | — | 7 (31.82) | — | | | | | |
| F/F | 12 (28.57) | — | 14 (63.63) | — | | | | | |
| FCGR3A V | 30 (71.43) | — | 8 (36.36) | — | 4.23 | 0.014 | 0.675 | 4.95 | 0.043 |
| carriers | | | | | [1.43-13.42] | | [0.551-0.799] | [1.07-27.48] | |

Table 1: Parameters influencing the lymphocytosis caused by rituximab in monotherapy in 68 patients with chronic lymphoid leukemia (CLL) The group of patients having a lymphocyte inhibition of less than 90% was used as a reference for odds ratios.

### Example 2

The inventors have also proposed a simple and quick method for analyzing the frequency of regulator B-cells. This method was compared to the reference method, which is based on the intra-cytoplasmic detection of the IL-10 synthesis influx cytometry.

The method consists of a first step for enriching the population of B lymphocytes, which normally make up the great minority among leukocytes, by negative selection from a peripheral blood sample. The blood is collected on a phase separator tube (of the CPT vacutainer BD type). Alternatively, the blood can be transferred from a primary tube containing an anticoagulant to the phase separator tube.

A depletion cocktail of the RosetteSep StemCell type is added to the blood sample in order to enrich the leukocyte phase with B lymphocyte. At the end of this step comprising an incubation, a separation by centrifugation and two cell wash centrifugations, a population containing more than 90% B lymphocytes is obtained.

The cells are counted by visual counting (cells under microscopy) or by cytometry in order to prepare a concentration of 6 million cells per mL and placed in culture in a medium containing RPMI+10% fetal calf serum. The cells are stimulated by a cocktail of polyclonal activators, such as a mixture of lignant-histidine+antibody CPG and CD40 patterns. An unstimulated well is done in parallel. The culture is kept for 18 h to 48 h at 37° C. without intervention.

At the end of this incubation, the cell culture is centrifugated and the culture supernatant is collected and kept at −20° C. or tested immediately through an enzyme-linked immunoabsorbent assay (EIA) in order to determine the concentration of IL-10 secreted.

The data obtained compared to the reference method in cytometry shows that the method by EIA assay of the IL-10 secretion provides well-correlated results between the two methods. Thus, the cell samples containing a proportion of regulator B-cells greater than 5% are correctly identified (FIG. 5).

1.-11. (canceled)

12. An in vitro prognosis method for a therapeutic response, mediated by a therapeutic antibody, in a tumor sample taken from a patient with a blood disease, said antibody being an antibody depleting the cells of said blood disease,
the method comprising an in vitro determination of a quantity of B lymphocyte cells secreting interleukin 10 in said sample,
such that a patient from whom the tumor sample is taken, having the quantity of B cells secreting IL-10 below 5% of a total cell quantity of said sample, will have more than a 50% chance of experiencing a depletion of more than 90% of cells of said blood disease after treatment with said therapeutic antibody.

13. The prognosis method according to claim 12, wherein the blood disease is a blood disease whose cells express CD20 surface marker.

14. The prognosis method according to claim 12, characterized in that the blood disease is chosen from among B-cell chronic lymphoid leukemia (CLL), diffuse large B cell lymphomas and all of the histological variants expressing CD20, follicular lymphomas and mantle cell lymphomas, marginal zone lymphomas, MALT (mucosa-associated lymphoid tissue) lymphomas, Burkitt lymphomas, lymphoplasmacytic lymphomas, Waldenström's disease, B-cell prolymphocytic leukemia, and all unclassifiable CD20+ B-cell lymphomas.

15. The prognosis method according to claim 12, wherein the quantity of B-cells secreting IL-10 in the sample below 5% is measured by flux cytometry, after activating the secretion of IL-10, or by measuring a circulating IL-10 level in serum.

16. The prognosis method according to claim 15, wherein the quantity of B-cells secreting IL-10 is measured by flux cytometry, after activating the secretion of IL-10 and deactivating this secretion.

17. The prognosis method according to claim 12, wherein said antibody is an IgG1 or IgG3 antibody.

18. The prognosis method according to claim 17, wherein said antibody is a CD20 antibody.

19. The prognosis method according to claim 17, wherein said antibody is a rituximab antibody.

20. An in vitro prognosis method for a therapeutic response, mediated by a therapeutic antibody, in a tumor sample taken from a patient with a blood disease, said antibody being an antibody depleting cells of said blood disease, the method comprising an in vitro determination in the cells of said sample:
of the amino acid in position 176 of a sequence of the FcγRIIIa receptor, as shown by sequence SEQ ID NO: 1, or in position 158 of the sequence of the FcγRIIIa receptor, as shown by sequence SEQ ID NO: 2, and
of a quantity of B lymphocyte cells secreting interleukin 10 before a treatment, such that a patient, from whom the tumor sample is taken,
having a valine in position 176 of the sequence of the FcγRIIIa receptor, as shown by sequence SEQ ID NO: 1, or in position 158 of the sequence of the FcγRIIIa receptor, as shown by sequence SEQ ID NO: 2, and
having a quantity of B cells secreting IL-10 in the sample below 5% of the total cells of the sample will have more than a 50% chance of experiencing a depletion of more than 90% of the cells of said blood disease after treatment with said therapeutic antibody.

21. The prognosis method according to claim 20, wherein the blood disease is a blood disease whose cells express CD20 surface marker.

22. The prognosis method according to claim 20, characterized in that the blood disease is chosen from among B-cell chronic lymphoid leukemia (CLL), diffuse large B cell lymphomas and all of the histological variants expressing CD20, follicular lymphomas and mantle cell lymphomas, marginal zone lymphomas, MALT (mucosa-associated lymphoid tissue) lymphomas, Burkitt lymphomas, lymphoplasmacytic lymphomas, Waldenström's disease, B-cell prolymphocytic leukemia, and all unclassifiable CD20+ B-cell lymphomas.

23. The prognosis method according to claim 20, wherein the quantity of B-cells secreting IL-10 in the sample below 5% is measured by flux cytometry, after activating the secretion of IL-10, or by measuring a circulating IL-10 level in serum.

24. The prognosis method according to claim 23, wherein the quantity of B-cells secreting IL-10 is measured by flux cytometry, after activating the secretion of IL-10 and deactivating this secretion.

25. The prognosis method according to claim 20, wherein the in vitro determination of the amino acid in position 176 of the sequence of the FcγRIIIa receptor, as shown by sequence SEQ ID NO: 1, or in position 158 of the sequence of the FcγRIIIa receptor, as shown by sequence SEQ ID NO: 2, is done by polymerase chain reaction (PCR).

26. The prognosis method according to claim 20, where said antibody is an IgG1 or IgG3 antibody.

27. The prognosis method according to claim 26, wherein said antibody is a CD20 antibody.

28. The prognosis method according to claim 26, wherein said antibody is a rituximab antibody.

29. A kit for the in vitro prognosis of a patient with a blood disease who may be treated with a therapeutic antibody, comprising:
a detector for detecting an amino acid in position 176 of a sequence of a FcγRIIIa receptor, as shown by sequence SEQ ID NO: 1, or in position 158 of the sequence of the FcγRIIIa receptor, as shown by sequence SEQ ID NO: 2,
means for determining quantity of B lymphocyte cells secreting IL-10, and
one or several control samples.

30. The prognosis kit according to claim 29, in which the detector for detecting the amino acid in position 176 of a sequence of a FcγRIIIa receptor, as shown by sequence SEQ ID NO: 1, or in position 158 of the sequence of the FcγRIIIa receptor, as shown by sequence SEQ ID NO: 2, comprise oligonucleotides with sequences SEQ ID NO: 3, SEQ ID NO: 4 and SEQ ID NO: 5.
